(19) United States
(12) Patent Application Publication
(10) Pub. No.: US 2026/0325224 A1
(43) Pub. Date: Oct. 22, 2026

(54) METHOD AND COMPUTER PROGRAMS FOR GUIDING AN IMPLANTABLE DEVICE DURING A CLINICAL INTERVENTION

(71) Applicant: MENTICE INC., Chicago, IL (US)

(72) Inventors: Héctor FERNÁNDEZ MARTÍNEZ, Barcelona (ES); Nieves MONTES GÓMEZ, Sant Quirze del Valles (ES); Arià JAIMEJUAN, Lleida (ES); Laura OBRADÓ GARCÍA, Granollers (ES)

(21) Appl. No.: 19/699,998

(22) Filed: Jun. 5, 2026

Related U.S. Application Data
(63) Continuation of application No. PCT/US2024/051859, filed on Oct. 17, 2024.
(60) Provisional application No. 63/607,341, filed on Dec. 7, 2023.

(51) Int. Cl. A61B 34/20; A61B 34/10; G06T 7/246

(52) U.S. Cl. CPC A61B 34/20; A61B 34/10; G06T 7/251; A61B 2034/105; A61B 2034/107; G06T 2207/30101; G06T 2207/30172

(57) ABSTRACT

A method and computer programs for guiding an implantable device during a clinical intervention are disclosed. The method comprises a) generating a 3D vasculature model; b) defining a target deployment plan of the device inside the 3D vasculature model; c) obtaining a configuration parameter of the device; d) receiving a live image feed corresponding to a real-time deployment of the device within the 3D vasculature model; e) segmenting the deployed device in the live image feed, and obtaining a point-to-point longitudinal trajectory of the deployed device; f) extracting the same configuration parameter as in step c) from the deployed segmented device; g) determining the trajectory of the deployed segmented device on the generated 3D vasculature model; h) comparing the determined trajectory and configuration parameter of the deployed segmented device with the feasible trajectory and configuration parameter; and i) assessing closeness between the defined target deployment plan and the real-time deployment based on the comparison.

### CROSS-REFERENCE TO RELATED APPLICATION(S)

The present application is a continuation of International Application No. PCT/US2024/051859, filed Oct. 17, 2024, which claims priority to, and the benefit of, U.S. Provisional Patent Application No. 63/607,341, filed on Dec. 7, 2023. The content of the aforementioned application is herein incorporated by reference in its entirety.

### TECHNICAL FIELD

The present invention is directed, in general, to (computer-aided) methods for implantable medical devices. In particular, the invention refers to a (computer-implemented) method and computer programs for guiding an implantable device during a clinical intervention.

### BACKGROUND OF THE INVENTION

X-ray C-arm systems are often used for the treatment of vascular diseases. In this procedure, the operator introduces a catheter inside the vessels of a treated patient, injects a contrast agent (a liquid that absorbs X-ray energy and thus becomes shadowy to the X-ray images being acquired), and navigates the anatomy of the patient to reach the specific location to be treated. Once at the location, the operator performs different protocols to acquire a detailed image of the local vascular anatomy, either 2D or 3D, via tomographic reconstruction.

An aneurysm is a dilation of the vessel wall, which might rupture and lead to a hematoma. This kind of vascular disease can be treated endovascularly, reaching the location with a catheter to then release an endovascular device, such as a stent, coils, or an intrasaccular device. Some stents, such as flow diverters, are made of interwoven wires, which can adopt different shapes and positions according to how the operator maneuvers the device while deploying it. Achieving a specific position and path of the device can have different effects on the disease, and some configurations can be hard to achieve. Particularly, when the aneurysm is large or fusiform, the path followed by the device, its diameter, and porosity at every given location, among others, which can be manipulated by the operator. In these situations, planning the case is crucial to achieve the best possible outcome of the treatment.

In many situations, the anatomy is complex making the selection of the desired device not trivial. This selection can be done by measurement of the anatomy, by simulation, or even using plastic/silicone phantoms that capture the complexity of the anatomy and are used to test and train the case before performing the treatment on the patient.

Endovascular implantable devices can be of different types, sizes and shapes. Some devices are placed inside the vessel (these are called endovascular implantable devices) while others, for instance for the treatment of intracranial aneurysms, are designed to be placed inside the sac of the aneurysm (and are called intrasaccular devices).

Determining the device size and length of the implantable device to be used is highly dependent on the patient's anatomy. For this reason, the use of simulation can be crucial. The simulation of aneurysm treatment for device size selection uses as input the 3D anatomy of the patient, typically acquired using a 3DRA, CTA, MRA or other modality with contrast for clear visualization of the vascular anatomy. From this anatomy, a 3D model of the region of interest to be treated is produced. The operator will then simulate devices of varying diameters and length according to the treatment's objectives. After choosing the ideal device length and diameter, the operator can assess different configurations of device maneuvering by changing the path of the device medial axis (centerline) or by compressing the mesh of the device to maximize the device diameter inside the vessel lumen free space. The combination of the device brand, size (length and diameter), position, and maneuvering, results in a treatment plan for that patient. All the information obtained from the treatment planning can be recorded to be later, during the actual treatment, presented to the operator as guidance to the treatment that the operator wants to target.

Another way of planning a case is by preparing the case by means of a plastic or silicone model of the patient anatomy. In this case, after extracting the anatomy of the patient from the 3D image, the anatomy is reconstructed. This reconstructed anatomy is then 3D printed, cast, or elaborated in any other way known in the state of the art, producing a physical model of the vessel and its disease. With this physical model of the patient's anatomy, the operator will navigate the model with a catheter and physically deploy one or more devices inside the anatomy to find the device that is adequate for that anatomy and disease. This process of device release can be guided using an X-ray C-Arm, or, when the model is transparent, any kind of optical camera or microscope to help the operator navigate the anatomy. The images obtained in the process can be recorded, as well as the view angle used to approach the treatment. This information can then be presented to the operator during the treatment to be used as guidance to reach the target treatment as planned.

The mentioned implantable endovascular or intrasaccular devices mentioned before are typically presented by the manufacturers inside a microcatheter, a sheath or sleeve, in a collapsed form. These devices present different radiopaque markers, as well as being made of a radiopaque material themselves, making them visible under fluoroscopy. This makes it possible for the devices to be properly visible and tractable inside the body and during the treatment.

Each one of these treatment planning alternatives provide useful information for the guidance of the treatment, such as the position of the device relative to the anatomy, its location, whether the device should be maneuvered while extracting it from the delivery system, among others.

In the state-of-the-art, different tools and techniques supporting the visualization of the device during the treatment and guide the operator in the process of reaching a specific location of a device with a known size device are known. For instance, U.S. Pat. No. 8,060,186B2, discloses a method for guiding stent deployment during an endovascular procedure, which includes providing a virtual stent model of an real stent that specifies a length, diameter, shape, and placement location of the real stent, where the stent includes radio-opaque end markers, and the virtual stent model includes virtual end markers that are projected onto the fluoroscopic images, projecting an outline and shape taken from the virtual stent model onto a 2D DSA image of a target lesion, manipulating a stent deployment mechanism to navigate the real stent to the target lesion while simultaneously acquiring real-time 2D fluoroscopic images of the stent navigation, and overlaying each fluoroscopic image on the 2D DSA image having the projected virtual stent model image, where the 2D fluoroscopic images are acquired from a C-arm mounted X-ray apparatus, and updating the projection of the virtual stent model onto the fluoroscopic images whenever a new fluoroscopic image is acquired or whenever the C-arm is moved, where the stent is aligned with the virtual stent model by aligning the stent end markers with the virtual end markers. However, such tools do not provide guidance on how to maneuver the device to reach a specific configuration of the device or a specific maneuvering to reach the desired shape, path and expansion of the device.

New and improved methods for guiding an implantable device during a clinical intervention are therefore needed.

### DESCRIPTION OF THE INVENTION

To that end, embodiments of the present invention provide a method for guiding an implantable device during a clinical intervention. The method comprises performing by one or more processors the following steps: a) generating a 3D vasculature model of a patient, the generated 3D vasculature model comprising a virtual 3D vasculature model or a physical 3D vasculature model; b) defining a target deployment plan of the implantable device inside the generated 3D vasculature model of the patient by obtaining at least one feasible trajectory of the implantable device within the generated 3D vasculature model; c) obtaining at least one configuration parameter of the implantable device; d) receiving, from a medical imaging device, a live image feed corresponding to a real-time partial or total deployment of the implantable device within the 3D vasculature model; e) segmenting the partially or totally deployed implantable device in the received live image feed using a segmentation technique, and obtaining a point-to-point longitudinal trajectory of the partially or totally deployed implantable device; f) extracting, along the obtained point-to-point longitudinal trajectory, the same at least one configuration parameter as in step c) from the partially or totally deployed segmented implantable device; g) determining the trajectory of the partially or totally deployed segmented implantable device on the generated 3D vasculature model by mapping the at least one feasible trajectory with each point of the point-to-point longitudinal trajectory; h) comparing, when the partially or totally deployed segmented implantable device, or at least a portion thereof, is in contact with a vessel of the generated 3D vasculature model, the determined trajectory and configuration parameter of the partially or totally deployed segmented implantable device with the at least one feasible trajectory and configuration parameter of steps b) and c); and i) assessing a closeness between the defined target deployment plan and the real-time deployment based on a result of the comparison. If in step d) the implantable device is partially deployed, the method comprises repeating steps d) to i) until a complete deployment of the implantable device.

According to the invention, the implantable device can be either an endovascular device or an intrasaccular device, among other implantable devices.

In some embodiments, the virtual 3D vasculature model is generated by segmenting one or more medical images of the patient, where the one or more medical images are acquired from the patient prior to the clinical intervention and comprise contrast.

In some embodiments, the physical 3D vasculature model is generated by segmenting one or more medical images of the patient, obtaining a virtual 3D vasculature model as a result, the one or more medical images being acquired from the patient prior to the clinical intervention and comprising contrast, and printing a physical replica of the vasculature with a given material using the virtual 3D vasculature model.

In some embodiments, the at least one feasible trajectory of the partially or totally deployed implantable device within the generated 3D vasculature model and the point-to-point longitudinal trajectory of the partially or totally deployed implantable device are obtained by obtaining at least one centerline within the virtual 3D vasculature model.

In some embodiments, the at least one centerline is obtained by considering a modified inscribed radius R′(x) function, and by modifying the potential field defined on a 3D space of the generated 3D vasculature model over which the centerline trajectory is computed.

In some embodiments, the at least one configuration parameter is obtained from a simulation of the implantable device. Alternatively, in other embodiments, it is obtained from morphological parameters of the vessel.

In some embodiments, the configuration parameter comprises a point to point trajectory, including end-point locations, of the implantable device; a cross-section expansion of the implantable device defined over a trajectory thereof; and/or a local porosity defined over a surface of the implantable device.

In some embodiments, the result of the comparison comprises a continuous or a discrete score.

In some embodiments, the configuration parameter comprises a point to point trajectory that is obtained as the medial axis of the patient vasculature.

In some embodiments, the point-to-point longitudinal trajectory is obtained as the medial axis of the partially or totally deployed segmented implantable device.

In some embodiments, the medical imaging device comprises a 2D scanner or a 3D image reconstruction device.

In some embodiments, the method uses real-time imaging (e.g., X-ray or other methods) of the patient's anatomy from a 2D scanner or 3D reconstruction to guide device placement during an intervention. When a target configuration from pre-interventional planning is available, the partially deployed device is compared against this configuration. The live feed can be compared to the target deployment obtained from the virtual or physical pre-treatment planning models. The invention assesses key morphological parameters such as: device position (both distal and proximal), device expansion, longitudinal trajectory of the device, comparison of vessel and device cross-section perimeters, percentage of apposition (device perimeter/vessel perimeter), device porosity.

Other embodiments of the invention that are disclosed herein also include a system and software programs to perform the method embodiment steps and operations summarized above and disclosed in detail below. More particularly, a computer program product is one embodiment that has a computer-readable medium including computer program instructions encoded thereon that when executed on at least one processor in a computer system causes the processor to perform the operations indicated herein as embodiments of the invention.

Therefore, present invention provides real-time guidance during device deployment based on pre-interventional planning. This includes selecting the local surface configuration of the device and assisting with user navigation.

It allows for the assessment of the target configuration, as determined during pre-interventional planning, by comparing virtual or physical device deployments with a virtual or physical model of the patient's anatomy. These models can be digitally created or physically fabricated (e.g., 3D printed) and are mapped to a live image feed. Users can explore different configurations during planning to account for various scenarios, with configurations influenced by morphological changes observed during deployment.

In some embodiments, the invention offers live evaluation of the endovascular procedure, tracking the device's morphological parameters as seen in the image feed during deployment.

A quantitative assessment of deployment quality is provided, comparing it to pre-intervention plans or, in cases where planning is unavailable, to the patient's vascular morphology.

### BRIEF DESCRIPTION OF THE DRAWINGS

The previous and other advantages and features will be more fully understood from the following detailed description of embodiments, with reference to the attached figures, which must be considered in an illustrative and non-limiting manner, in which:

FIG. 1 is a flow chart illustrating an embodiment of a method for guiding an implantable device during a clinical intervention.

FIGS. 2A-2B graphically illustrate how the centerline in the 3D vasculature model is obtained, according to an embodiment.

FIGS. 3A-3B illustrate a triangle function and sawtooth function, respectively, representing the shape of R′(x).

FIG. 4A provides a graphical representation of an XY plane cross-section of a vessel model, specifically cutting through the aneurysm sac. FIG. 4B graphically shows several R′(x) functions, each corresponding to different selections of the UB parameter. These functions are evaluated within the same XY plane of the vessel as shown in FIG. 4A.

FIG. 5 graphically illustrates the target device deployment in a physical 3D vasculature model, according to an embodiment.

FIGS. 6A-6C graphically illustrate an embodiment of steps 104-108 of the method of FIG. 1. From the life image feed during device deployment (FIG. 6A), segment the partially deployed device (FIG. 6B). A mapping between planned and deployed device centerlines is used to compare centerline trajectories and/or morphological parameters (FIG. 6C).

### DETAILED DESCRIPTION OF PREFERRED EMBODIMENTS

The present invention provides a (computer implemented) method for guiding an interventional radiologist in the process of delivering an endovascular implantable device inside a vessel while trying to target a specific device position and configuration that has been obtained during a treatment planning stage. In this process, medical images dynamically acquired during a treatment itself are processed by a computer to evaluate if the treatment planning is being achieved by the interventionist. This method is primarily thought to be used during the treatment of intracranial aneurysms with braided devices (such as flow diverters, stents, and intrasaccular devices). Still, a person skilled in the art of computer modelling and medical imaging can find different applications to use this methodology other than these ones. This technique is to be complemented by some methodology for planning the treatment, where a target treatment, device position and device configuration is obtained and then, during the treatment itself, the operator will perform the necessary operations to achieve that position guided by the methodology proposed by the present invention.

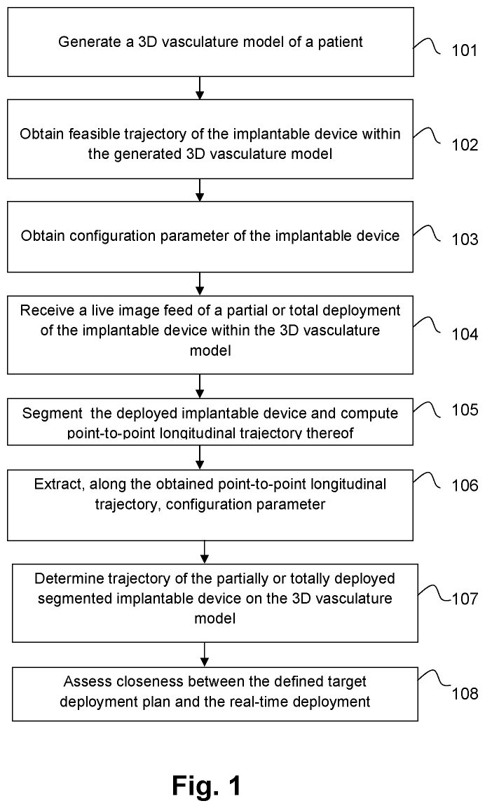

FIG. 1 is a flow chart illustrating an embodiment of the proposed method. According to this embodiment, the first step of the method comprises performing a virtual or physical device target deployment plan inside a virtual 3D vasculature model or a physical 3D vasculature model. To that end, in step 101, a 3D vasculature model of the patient vasculature, including the virtual 3D vasculature model or the physical 3D vasculature model, is generated, and then, in step 102, at least one feasible trajectory of the implantable device within the generated 3D vasculature model is obtained.

According to the invention, the 3D vasculature model can be generated using medical images (e.g. DICOM images, among others) of the patient taken prior to the intervention, with contrast to highlight the vessel lumen. The 3D vasculature model can be obtained using off-the-shelf segmentation methods (e.g. thresholding). When working with the virtual 3D model, this is obtained by just segmenting the medical images. When working with the physical 3D model, the virtual 3D model is used to print a physical replica of the vasculature in a suitable material, e.g. silicone, where the user can perform device deployment with techniques similar to those used when deploying the implantable device on a patient.

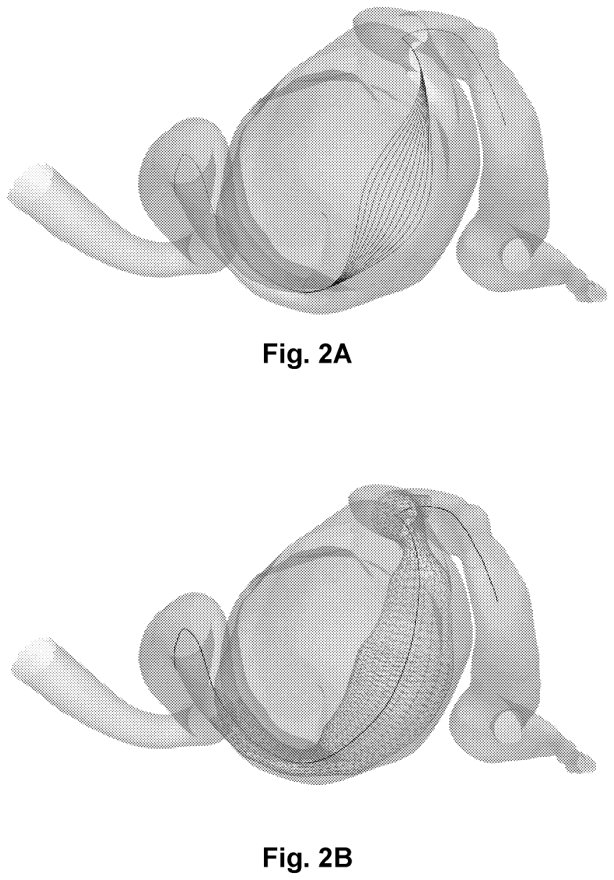

Within the virtual 3D vasculature model, the centerline of the vessel is calculated, representing the medial axis that maximizes the distance to the vessel wall. As shown in FIG. 2A, multiple centerlines may be generated, each corresponding to a different potential device trajectory influenced by user manipulation during deployment. These trajectories directly affect the device's configuration parameters, such as expansion and porosity, allowing the user to choose the trajectory best suited for the desired deployment outcome.

In a particular embodiment, the various centerline trajectories are computed by modifying the potential field defined on 3D space over which the centerline trajectory is computed. In general, the vessel medial axis is defined as the parametric path C(s) that minimizes the energy functional, as described in “Patient-Specific Modeling of Geometry and Blood Flow in Large Arteries (PhD thesis)”, Antiga Luca, 2002, Politecnico di Milano:

$$E = {\int }_{0 = {C}^{- 1} ( {p}_{0} )}^{L = {C}^{- 1} ( {p}_{f} )} F ( C ( s ) ) d s$$

where F(C(s)) is a field defined in 3D space and referred to as the centering potential. Usually, this centering potential is taken as the inverse of the maximum inscribed sphere:

$$F ( x ) = \frac{1}{R ( x )} ,$$

which can lead to distorted centerlines in the presence of large lesions such as aneurysms, where the most centered centerline is far from the path that would have been computed if there had not been an aneurysm in the vessel morphology.

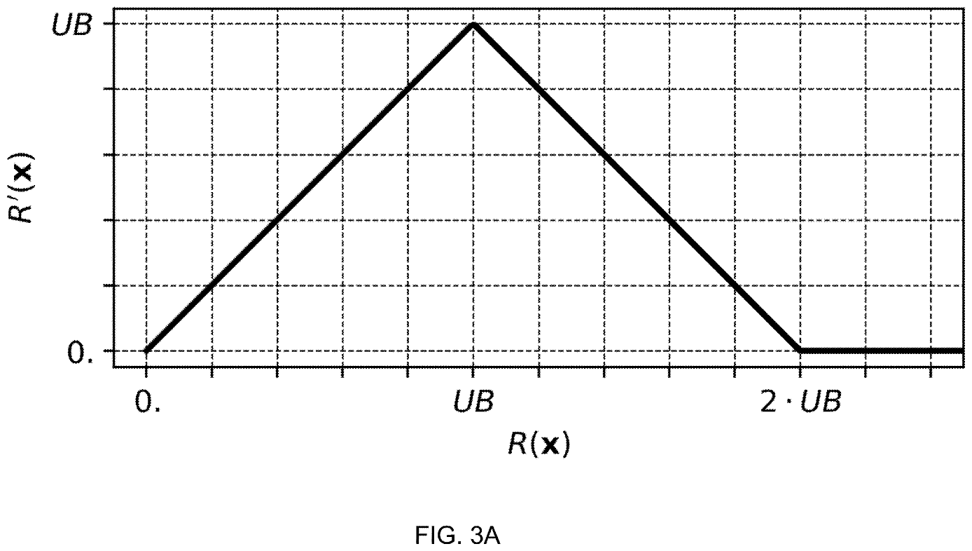

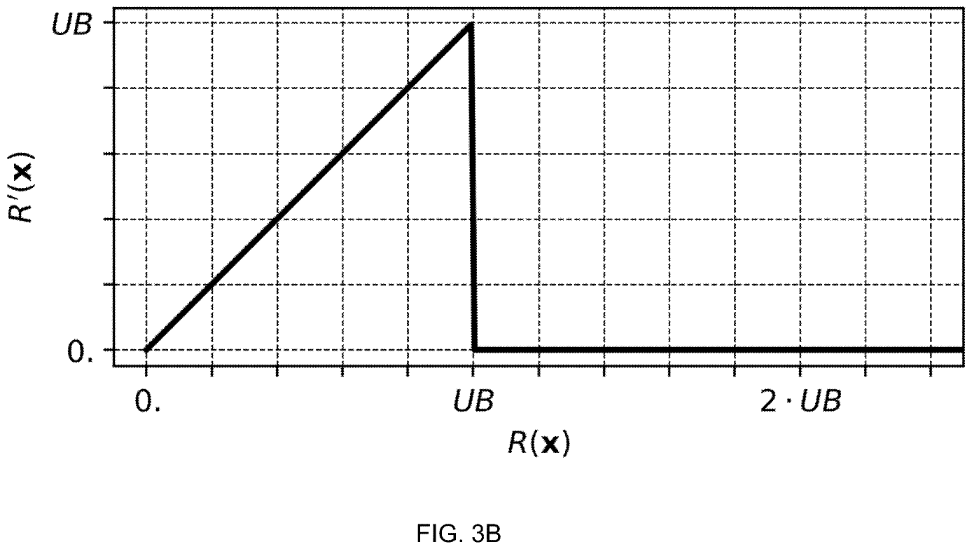

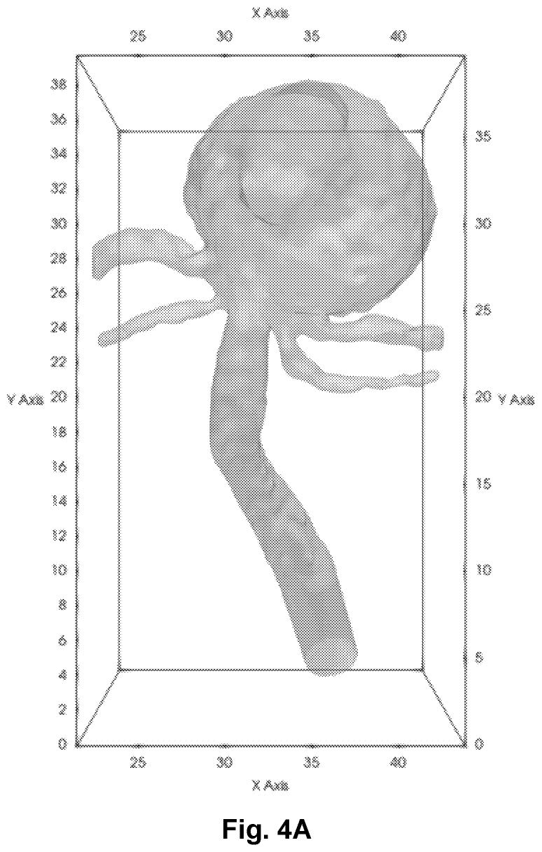

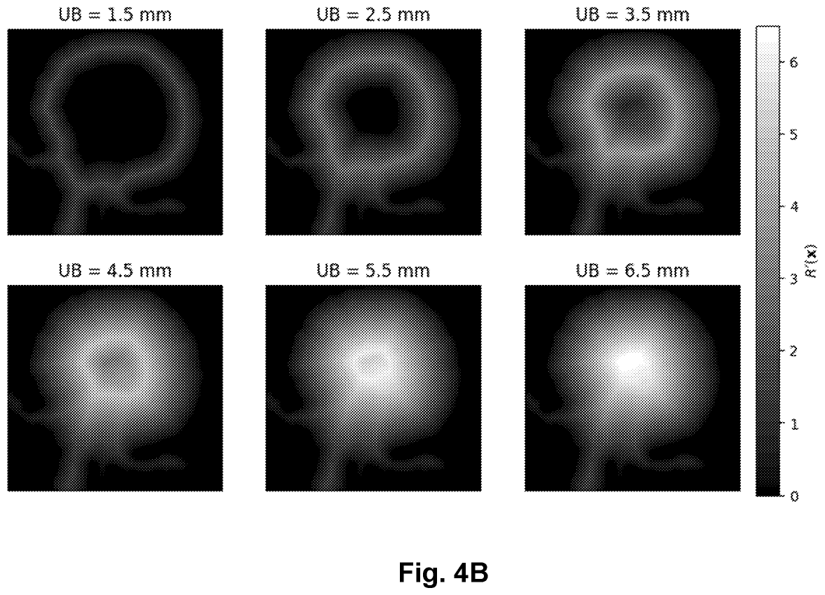

Hence, in this embodiment, the centerline paths are computed by introducing a modified inscribed radius R′(x) function such that R′(x)=R(x) if R(x)<Upper Bound (UB) and R′(x)≤R(x) otherwise. Such conditions have the effect of pushing the centerline away from regions where the inscribed radius is unrealistically large, such as is the case in aneurysms. The shape of R′(x) can be, but is not limited to, a triangle function (FIG. 3A), a sawtooth function (FIG. 3B) or any other function. Finally, for every choice of the UB parameter, a different centerline path is obtained. As an example, FIG. 4B shows several R′(x) functions by choice of UB parameter, evaluated on an XY plane of the vessel model from FIG. 4A cutting through the aneurysm sac.

In some embodiments, to improve computational efficiency, the centerline paths are not calculated one by one, but rather a baseline centerline path is extracted (which uses the unmodified inscribed radius R(x)). Then, a correction segment is identified as the centerline segment that goes through the aneurysm region. This segment is then discarded and re-calculated using, for example, the modified inscribed radius presented above. The method to identify the correction segment includes, but is not limited to, manual identification (e.g. the user identifies the endpoints of the centerline that should be corrected), automatic identification (e.g. using a trained machine learning model that tags the endpoints) or a combination of the previous (e.g. a machine learning model makes a proposal for the endpoints but allows the user to refine the selection).

Each centerline path leads to a deployment plan, which consists of a representation of the deployed implantable device. A deployed implantable device can be characterized by its final length and position (e.g. computed according to patent U.S. Pat. No. 10,176,566B2), its expansion along the centerline (e.g. computed from the inscribed radius along the centerline) and/or its surface porosity (e.g. computed according to patent U.S. Pat. No. 10,521,553B2).

In FIG. 2B, an example of a selected centerline is illustrated. This centerline determines the final device configuration, which is particularly defined by factors like the distal and proximal point locations, stent trajectory, expansion, and device porosity.

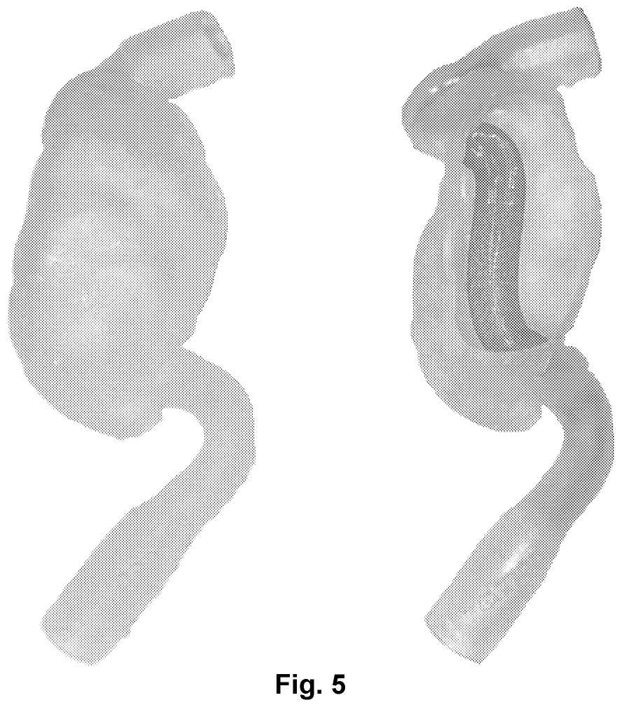

In FIG. 5, the target implantable device deployment and corresponding centerline(s) are obtained on a physical 3D vasculature model of the vasculature, around the area of interest.

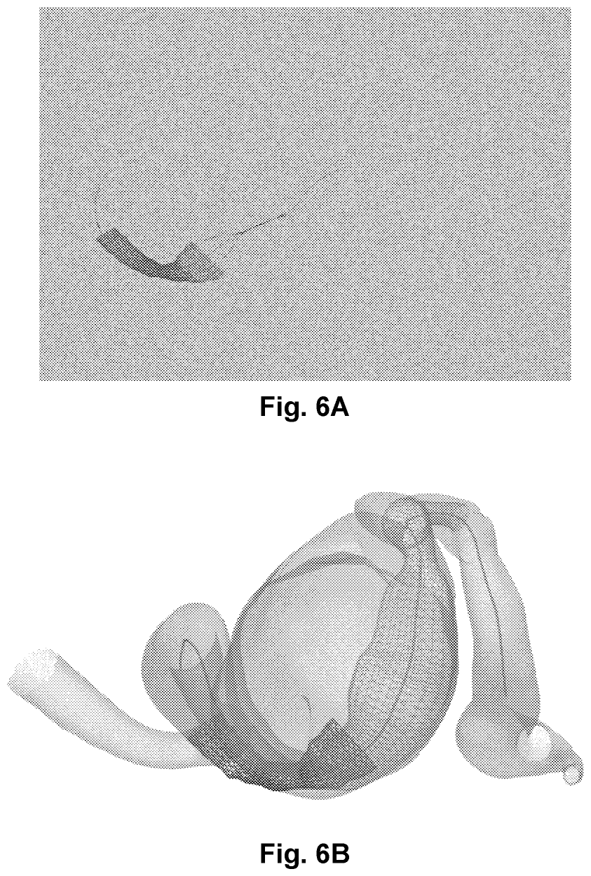

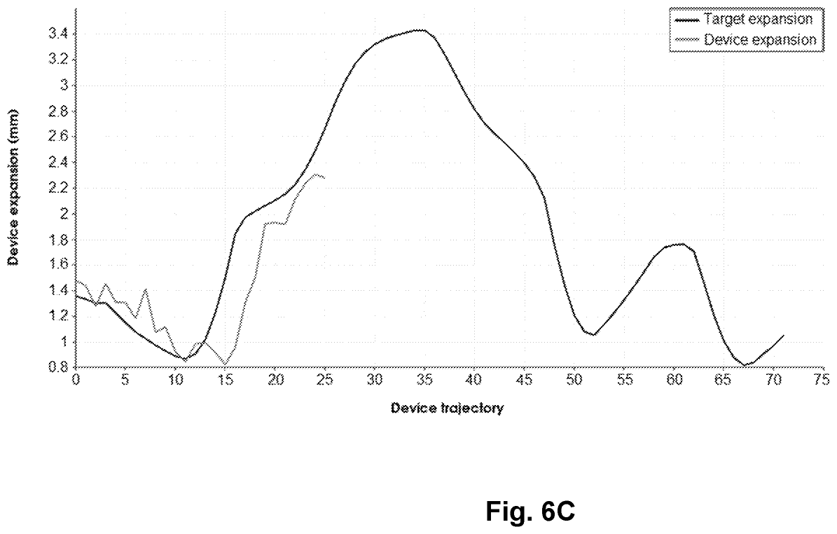

Referring back to FIG. 1, in step 103, the relevant parameters or combination of parameters describing the implantable device configuration are defined. These parameters may include the point-to-point trajectory (e.g., end-point locations), cross-section expansion along the trajectory, local porosity across the implantable device surface, and others. The user selects the most critical configuration parameters for evaluating the final deployment, focusing on those most pertinent to the success of the procedure. Then, in step 104, a live feed (see FIG. 6A for a visual representation thereof) obtained from a 2D scan, or 3D reconstruction, during device deployment is used to segment the (partially or totally) deployed implantable device and extract its point-to-point longitudinal trajectory (step 105). The segmentation and device trajectory extraction are analogous to that performed before.

In some embodiments, in step 103, the relevant parameters or combination of parameters describing the device configuration can be replaced by relevant morphological parameters of the vessel around the area of interest.

In a particular embodiment, the point-to-point longitudinal trajectory is obtained as the medial axis of the (partially or totally) deployed segmented implantable device.

In a particular embodiment, in step 105, the segmentation of the (partially or fully) deployed implantable device is obtained from the live feed during patient intervention. Also, the segmentation of the (partially or fully) deployed implantable device can be automated by setting a priori the parameters that define the segmentation process.

From the (partially or totally) deployed segmented implantable device, the method then extracts, in step 106, the same set of relevant morphological parameters as described in step c, along the device trajectory including visible end-points, and determines, in step 107, the trajectory of the (partially or totally) deployed implantable device, from step 106, on the virtual or physical 3D vasculature model, resulting in a point-to-point mapping from the (partially or totally) deployed implantable device trajectory to the planning device trajectory.

In some embodiments, in step 107, the location of the (partially or totally) segmented implantable device on the patient's virtual or physical vasculature model can be determined through automatic or manual alignment. Likewise, in some embodiments, step 107 can also involve mapping the (partially or totally) deployed segmented implantable device's trajectory to the virtual or physical device's target trajectory, point by point.

Finally, in step 108, the method assesses the proximity/closeness between the defined target deployment plan and the real-time deployment by comparing the trajectory and relevant morphological parameters of the (partially or fully) deployed implantable device with those from the virtual or physical deployment plan from steps 101 and 102. This comparison is only valid when the (partially or totally) deployed segmented implantable device, or at least part of it, is in contact with the vessel in the 3D vasculature model. The comparison is performed point-by-point along the matched device trajectories, providing a qualitative visualization for the user. This allows the user to see the key parameters of their planned deployment alongside the corresponding parameters of the real-time partially deployed implantable device.

Also in step 108, on embodiments on which the target deployment has been defined from morphological parameters of the vessel, the comparison between the real-time deployment and the target deployment can be used as an indicator of vessel deformation due to the intervention.

In some embodiments a score can be defined to assess said proximity/closeness. The score can be defined as a distance function between planned device and real-time segmented device salient parameters. For instance, it can be provided as a continuous grade (e.g. a mark from 0 to 100) or as a discrete grade (e.g. “excellent”, “good”, “OK”, “bad”). In some embodiments, the score can be generated from a combination of trajectory deviations and differences in relevant morphological parameters.

Note that if in step 104 the implantable device is partially deployed, steps 105-108 are repeated until a complete deployment of the implantable device.

The embodiments of the present invention can be implemented in various forms of hardware, software, firmware, or a combination thereof. The present invention can be implemented in software as an application program tangible embodied on a computer readable program storage device. The application program can be uploaded to, and executed by, a machine comprising any suitable architecture.

A machine-readable medium may take many forms, including but not limited to, a tangible storage medium, a carrier wave medium or physical transmission medium. Non-volatile storage media include, for example, optical or magnetic disks, such as any of the storage devices in any computer(s), or the like, which may be used to implement the system or any of its components shown in the drawings. Volatile storage media may include dynamic memory, such as a main memory of such a computer platform. Tangible transmission media may include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media may include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

The one or more processors may include one or more central processing units (CPUs), one or more microprocessors, one or more microcontrollers, one or more controllers, one or more complex instruction set computing (CISC) microprocessors, one or more reduced instruction set computing (RISC) microprocessors, one or more very long instruction word (VLIW) microprocessors, one or more graphics processor units (GPU), one or more digital signal processors, one or more application specific integrated circuits (ASICs), and/or any other type of processor or processing circuit capable of performing desired functions. The one or more processing devices can be configured to execute any computer program instructions that are stored or included on the one or more computer storage devices including, but not limited to, instructions for executing the functions associated with the method.

In certain embodiments, the one or more processors are installed in computing devices that may represent mobile devices (e.g., smart phones, personal digital assistants, tablet devices, etc.), desktop computers, laptop computers, servers, etc.

Various aspects of the proposed method may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors, or the like, or associated modules thereof, such as various semiconductor memories, tape drives, disk drives and the like, which may provide storage at any time for the software programming.

The scope of the present invention is defined in the claims that follow.

1. A method for guiding an implantable device during a clinical intervention, the method comprising performing by one or more processors the following steps:
a) generating a 3D vasculature model of a patient, the generated 3D vasculature model comprising a virtual 3D vasculature model or a physical 3D vasculature model;
b) defining a target deployment plan of the implantable device inside the generated 3D vasculature model of the patient by obtaining at least one feasible trajectory of the implantable device within the generated 3D vasculature model;
c) obtaining at least one configuration parameter of the implantable device;
d) receiving, from a medical imaging device, a live image feed corresponding to a real-time partial or total deployment of the implantable device within the 3D vasculature model;
e) segmenting the partially or totally deployed implantable device in the received live image feed using a segmentation technique, and obtaining a point-to-point longitudinal trajectory of the partially or totally deployed implantable device;
f) extracting, along the obtained point-to-point longitudinal trajectory, the same at least one configuration parameter as in step c) from the partially or totally deployed segmented implantable device;
g) determining the trajectory of the partially or totally deployed segmented implantable device on the generated 3D vasculature model by mapping the at least one feasible trajectory with each point of the point-to-point longitudinal trajectory;
h) comparing, when the partially or totally deployed segmented implantable device, or at least a portion thereof, is in contact with a vessel of the generated 3D vasculature model, the determined trajectory and configuration parameter of the partially or totally deployed segmented implantable device with the at least one feasible trajectory and configuration parameter of steps b) and c); and
i) assessing a closeness between the defined target deployment plan and the real-time deployment based on a result of the comparison,
wherein if in step d) the implantable device is partially deployed, the method comprises repeating steps d) to i) until a complete deployment of the implantable device.

2. The method of claim 1, wherein the virtual 3D vasculature model is generated by:
segmenting one or more medical images of the patient, the one or more medical images being acquired from the patient prior to the clinical intervention and comprising contrast; or
segmenting one or more medical images of the patient, obtaining a virtual 3D vasculature model as a result, the one or more medical images being acquired from the patient prior to the clinical intervention and comprising contrast, and printing a physical replica of the vasculature with a given material using the virtual 3D vasculature model.

3. The method of claim 1, wherein the at least one feasible trajectory of the partially or totally deployed implantable device within the generated 3D vasculature model and the point-to-point longitudinal trajectory of the partially or totally deployed implantable device being obtained by obtaining at least one centerline within the virtual 3D vasculature model.

4. The method of claim 3, the at least one centerline being obtained by considering a modified inscribed radius R′(x) function, and by modifying a potential field defined on a 3D space of the generated 3D vasculature model over which a centerline trajectory is computed.

5. The method of claim 1, wherein the at least one configuration parameter is obtained from a simulation of the implantable device and/or from morphological parameters of the vessel.

6. The method of claim 1, wherein the configuration parameter comprises a point to point trajectory, including end-point locations, of the implantable device; a cross-section expansion of the implantable device defined over a trajectory thereof; and/or a local porosity defined over a surface of the implantable device.

7. The method of claim 1, wherein the configuration parameter comprises a point to point trajectory that is obtained as the medial axis of the patient vasculature.

8. The method of claim 1, wherein the result of the comparison comprises a continuous or a discrete score.

9. The method of claim 1, wherein the point-to-point longitudinal trajectory is obtained as the medial axis of the partially or totally deployed segmented implantable device.

10. The method of claim 1, wherein the medical imaging device comprises a 2D scanner or a 3D image reconstruction device.

11. The method of claim 1, wherein the implantable device comprises an endovascular device or an intrasaccular device.

12. A non-transitory computer readable medium including code instructions that when executed in a computer system implement the steps of:
a) generate a 3D vasculature model of a patient, the generated 3D vasculature model comprising a virtual 3D vasculature model or a physical 3D vasculature model;
b) define a target deployment plan of the implantable device inside the generated 3D vasculature model of the patient by obtaining at least one feasible trajectory of the implantable device within the generated 3D vasculature model;
c) obtain at least one configuration parameter of the implantable device;
d) receive, from a medical imaging device, a live image feed corresponding to a real-time partial or total deployment of the implantable device within the 3D vasculature model;
e) segment the partially or totally deployed implantable device in the received live image feed using a segmentation technique, and obtaining a point-to-point longitudinal trajectory of the partially or totally deployed implantable device;
f) extract, along the obtained point-to-point longitudinal trajectory, the same at least one configuration parameter as in step c) from the partially or totally deployed segmented implantable device;
g) determine the trajectory of the partially or totally deployed segmented implantable device on the generated 3D vasculature model by mapping the at least one feasible trajectory with each point of the point-to-point longitudinal trajectory;
h) compare, when the partially or totally deployed segmented implantable device, or at least a portion thereof, is in contact with a vessel of the generated 3D vasculature model, the determined trajectory and configuration parameter of the partially or totally deployed segmented implantable device with the at least one feasible trajectory and configuration parameter of steps b) and c); and
i) assess a closeness between the defined target deployment plan and the real-time deployment based on a result of the comparison,
wherein if in step d) the implantable device is partially deployed, steps d) to i) are repeated until a complete deployment of the implantable device.

13. The non-transitory computer readable medium of claim 12, wherein the virtual 3D vasculature model is generated by segmenting one or more medical images of the patient, the one or more medical images being acquired from the patient prior to the clinical intervention and comprising contrast.

14. The non-transitory computer readable medium of claim 12, wherein the physical 3D vasculature model is generated by:
segmenting one or more medical images of the patient, obtaining a virtual 3D vasculature model as a result, the one or more medical images being acquired from the patient prior to the clinical intervention and comprising contrast, and
printing a physical replica of the vasculature with a given material using the virtual 3D vasculature model.

15. The non-transitory computer readable medium of claim 12, wherein the at least one feasible trajectory of the partially or totally deployed implantable device within the generated 3D vasculature model and the point-to-point longitudinal trajectory of the partially or totally deployed implantable device being obtained by obtaining at least one centerline within the virtual 3D vasculature model.

16. The non-transitory computer readable medium of claim 12, wherein the at least one configuration parameter is obtained from a simulation of the implantable device and/or from morphological parameters of the vessel.

17. The non-transitory computer readable medium of claim 12, wherein the configuration parameter comprises a point to point trajectory, including end-point locations, of the implantable device; a cross-section expansion of the implantable device defined over a trajectory thereof; and/or a local porosity defined over a surface of the implantable device.

18. The non-transitory computer readable medium of claim 12, wherein the result of the comparison comprises a continuous or a discrete score.

19. The non-transitory computer readable medium of claim 12, wherein the configuration parameter comprises a point to point trajectory that is obtained as the medial axis of the patient vasculature.

20. The non-transitory computer readable medium of claim 12, wherein the point-to-point longitudinal trajectory is obtained as the medial axis of the partially or totally deployed segmented implantable device.
